(19) United States
(12) Patent Application Publication
(10) Pub. No.: US 2022/0252581 A1
(43) Pub. Date: Aug. 11, 2022

(54) LATERAL FLOW ASSAY FOR DETECTION OF MONENSIN

(71) Applicant: Huvepharma Inc., Peachtree City, GA (US)

(72) Inventors: Ryan L. Vander Veen, Lincoln, NE (US); Mary M. McIlhaney, Wahoo, NE (US); Thomas W. Campi, Fishers, IN (US)

(21) Appl. No.: 17/548,314

(22) Filed: Dec. 10, 2021

Related U.S. Application Data
(60) Provisional application No. 63/124,551, filed on Dec. 11, 2020.

(51) Int. Cl. G01N 33/53; G01N 33/543; B01L 3/00

(52) U.S. Cl. CPC G01N 33/5308; G01N 33/54388; G01N 2333/195; B01L 2300/069; B01L 3/5023

(57) ABSTRACT

Methods are provided for detecting the presence of monensin in animal feed using a competitive lateral flow assay. The methods allow for qualitative detection of monensin, as well as quantitative detection at a very high level of sensitivity, e.g., detection of 10 ppm or less in a feed sample. Competitive lateral flow assay strip devices are also provided for use in the methods.

### CLAIM OF PRIORITY

This application claims priority to U.S. Provisional Application No. 63/124,551, filed Dec. 11, 2020, its entire disclosure of which is incorporated by reference herein.

### BACKGROUND OF THE INVENTION

Monensin is a polyether antibiotic ionophore related to the crown ethers with a preference to form complexes with monovalent cations such as: Li⁺, Na⁺, K⁺, Rb⁺, Ag⁺, and Tl⁺. Monensin is able to transport these cations across lipid membranes of cells in an electroneutral (i.e. non-depolarizing) exchange, playing an important role as an Na⁺/H⁺ antiporter. Recent studies have shown that monensin may transport sodium ion through the membrane in both electrogenic and electroneutral manner. This approach explains the ionophoric ability and, consequently, the antibacterial properties of not only parental monensin, but also its derivatives that do not possess carboxylic groups. It blocks intracellular protein transport, and exhibits antibiotic, antimalarial and other biological activities. The antibacterial properties of monensin and its derivatives are a result of their ability to transport metal cations through cellular and subcellular membranes.

Monensin is broadly used in the animal health industry to medicate feed for cattle and poultry. However, because of its ionophore activity, which prevents unwanted bacterial and protozoa activity in growing animals, it can also have a deleterious effect on mammalian membranes. Monensin poisoning is well documented in animal health medicated feed and great care is taken to make sure that the monensin content is not too high for animals using medicated feed (see e.g., Chalmers (1981) Can. Vet. J. 22:21-22; Potter et al. (1984) J. Anim. Sci. 58:1499-1511; Gonzalez et al. (2005) Can. Vet. J. 46:910-912). The concentration of monensin is often utilized in the range of a few grams/ton to at most a few hundred grams/ton of feed depending on the species of animal, age, and production utilization. For example, dairy cattle require a different dose of monensin than beef cattle. Similarly, starter rations for young chicks is much different than for growing broilers or layers. Accordingly, feed mills utilized by the industry to produce medicated feed have a need to measure the precise amount of monensin in the feed. Moreover, when animals are removed from monensin for different production purposes, feed mills must take care to make sure that their milling equipment is free of monensin to ensure that there is not a deleterious effect of monensin in the feed for safety to the animal or animal product such as milk or eggs.

One method that is currently used to measure monensin in feed is high performance liquid chromatography (HPLC), which can be costly, time consuming, and interruptive to scheduling of feeding programs and use of labor/equipment. Accordingly, there is a need for a fast, dependable, cost-effective, and labor-efficient method to determine the presence of monensin in feed.

### SUMMARY OF THE INVENTION

The present disclosure provides methods and devices for a competitive lateral flow assay (LFA) for the detection of monensin, e.g., in animal feed. The methods and devices of the disclosure provide a rapid, cost-effective and labor-efficient approach for detecting monensin, while also allowing for very sensitive detection, e.g., a lower limit of detection of monensin of as little as 10 ppm (or even less) in animal feed. Moreover, the methods and devices allow for quantitative measurement of monensin as well as qualitative detection, thereby allowing animal feed users to have very precise control when using the LFA methods and devices for adjusting levels of monensin in the feed.

Accordingly, in one aspect, the disclosure pertains to a competitive lateral flow assay (LFA) strip device for detection of monensin in a liquid sample, the device comprising:

a sample pad;

a conjugate pad loaded with an anti-monensin-specific antibody (M antibody) conjugated to a detectable label and a control antibody (C antibody) conjugated with a detectable label; and

a membrane surface comprising a test line with immobilized monensin to which the M antibody binds and a control line with immobilized antigen to which the C antibody binds,

wherein a liquid sample applied to the sample pad flows first through the conjugate pad and then across the test line and control line of the membrane surface;

wherein binding of the M antibody to the test line results in a detectable signal and binding of the C antibody to the control line results in a detectable signal;

wherein presence of monensin in the liquid sample decreases the detectable signal at the test line relative to a sample lacking monensin; and

wherein the LFA strip device has a sensitivity of 10 ppm or less for detecting monensin in the liquid sample.

In one embodiment, the LFA strip device has a lower limit of sensitivity to detect 6-10 ppm, or 6.6-10 ppm, or 7-10 ppm, or 8-10 ppm, or 9-10 ppm of monensin. In certain embodiments, the LFA strip device has a lower limit of sensitivity to detect 10 ppm, 9 ppm, 8 ppm, 7 ppm, 6.6 ppm, 6 ppm, 5 ppm, 4 ppm, 3 ppm, or 2 ppm of monensin.

In one embodiment, the sample pad is a polyester fiber pad. In one embodiment, the conjugate pad is a chopped glass pad. In one embodiment, the membrane surface comprises a nitrocellulose membrane. Suitable materials for these components of the LFA strip device are described further herein.

In one embodiment, the M monensin specific antibody is conjugated to gold nanoparticles.

In one embodiment, the immobilized monensin at the test line is BSA-monensin, which is detectable by the monensin-specific M antibody.

In one embodiment, the C antibody is anti-chicken IgY antibody or anti-mouse IgG conjugated to gold nanoparticles and the immobilized antigen at the control line is chicken IgY or mouse IgG.

In one embodiment, the conjugate pad is loaded with the M antibody at a concentration of 25-50 ug/mL. In one embodiment, the conjugate pad is loaded with the M antibody at a concentration of 30-40 ug/mL. In one embodiment, the conjugate pad is loaded with the M antibody at a concentration of 30 ug/mL.

In one embodiment, the conjugate pad is loaded with 90-95% M antibody and 5-10% C antibody. In one embodiment, the conjugate pad is loaded with 90% M antibody and 10% C antibody. In one embodiment, the conjugate pad is loaded with 95% M antibody and 5% C antibody.

In another aspect, the disclosure pertains to a method of detecting monensin, e.g., in animal feed, using the LFA device of the disclosure. Accordingly, the disclosure provides a method of detecting the presence of monensin in animal feed, the method comprising:

(a) contacting (e.g., incubating, mixing, or suspending) a sample of the animal feed with a liquid extraction buffer to obtain a liquid sample;

(b) applying the liquid sample (e.g., a predetermined amount) to the sample pad of an LFA strip device of the disclosure;

(c) developing the LFA strip device for at least 3 minutes (i.e., allowing the liquid sample applied to the sample pad to flow through the conjugate pad and onto the membrane surface, across the test and control lines of the LFA strip device); and

(d) visually reading or quantitatively measuring the LFA strip device to thereby detect the presence of monensin in the animal feed.

In one embodiment, the LFA strip device is developed for at least 5 minutes.

In one embodiment, the liquid extraction buffer comprises an organic solvent, such as an alcohol. In one embodiment, the alcohol in the liquid extraction buffer is ethanol. In one embodiment, the liquid extraction buffer comprises phosphate buffered saline (PBS) with 0.5% Tween-20 and 10% ethanol. In one embodiment, the liquid extraction buffer is an aqueous buffer, such as phosphate buffered saline (PBS) with 1% Tween-20.

In one embodiment, the extraction buffer is compatible with or optimal for allowing binding to the M or C antibody as the liquid sample moves across the test strip.

In one embodiment, the animal feed is contacted with the extraction buffer for 20 minutes or less to obtain the liquid sample. In one embodiment, the animal feed is contacted with the extraction buffer for 5 minutes to obtain the liquid sample.

In one embodiment of the method, the LFA strip device is read quantitatively using a quantitative reader (e.g., a reader that quantifies the optical density (OD) generated at the test line of the LFA strip device).

### BRIEF DESCRIPTION OF THE FIGURES

FIG. 1 is schematic diagram of a representative competitive LFA strip device for detection of monensin.

FIG. 2 is a graph showing detection of various dilutions of unlabeled monensin in an inhibition ELISA.

FIG. 3 is a graph showing the monensin standard curve for the inhibition ELISA.

FIG. 4 is a graph showing detection of monensin in ethanol-extracted animal feed by inhibition ELISA.

FIG. 5 is a graph showing detection of monensin in PBS-extracted animal feed by inhibition ELISA.

FIG. 6A is a photograph of LFA strips loaded with 30 ug/mL of anti-monensin conjugate and tested with samples extracted from feed containing 6 g monensin/ton of feed.

FIG. 6B is a photograph of LFA strips loaded with 30 ug/mL of anti-monensin conjugate and tested with samples extracted from feed containing 9 g monensin/ton of feed.

FIG. 6C is a photograph of LFA strips loaded with 40 ug/mL of anti-monensin conjugate and tested with samples extracted from feed containing 6 g monensin/ton of feed.

FIG. 6D is a photograph of LFA strips loaded with 40 ug/mL of anti-monensin conjugate and tested with samples extracted from feed containing 9 g monensin/ton of feed.

FIG. 7 is a graph showing levels of monensin detected using LFA strips with feed samples containing 0, 6, 9 or 12 grams monensin/ton of feed. Results are described in units of optical density using a quantitative OD reader for each concentration of sample.

FIG. 8A is a graph showing levels of monensin detected using LFA strips with feed samples containing 0, 6, 9 or 12 grams monensin/ton of feed wherein the feed sample was extracted for 5 minutes. Results are described in units of optical density using a quantitative OD reader for each concentration of sample.

FIG. 8B is a graph showing levels of monensin detected using LFA strips with feed samples containing 0, 6, 9 or 12 grams monensin/ton of feed wherein the feed sample was extracted for 20 minutes. Results are described in units of optical density using a quantitative OD reader for each concentration of sample.

FIG. 9 is a photograph of LFA strips tested with samples extracted from feed containing 0, 2, 5, 10, and 15 ppm of monensin.

### DETAILED DESCRIPTION OF THE INVENTION

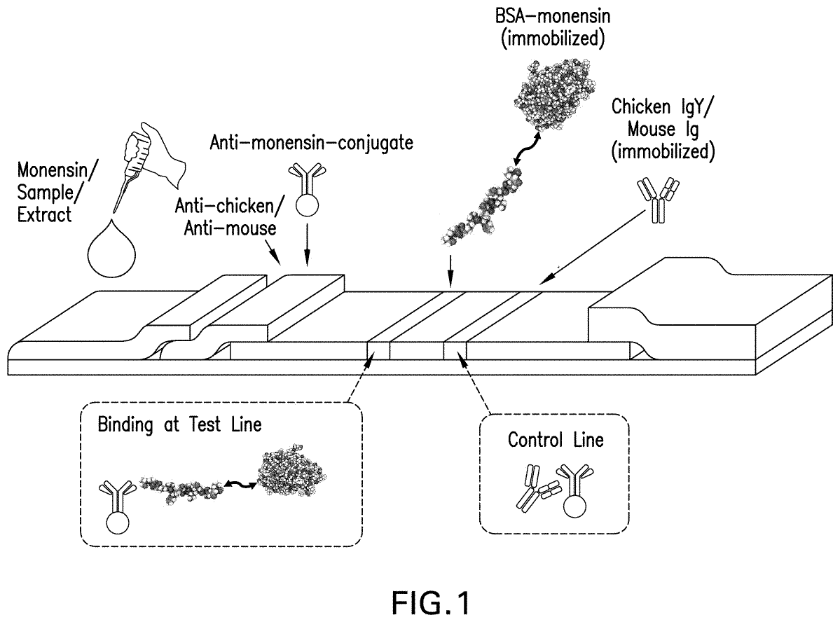

The disclosure provides methods and devices for a competitive lateral flow assay (LFA) for detection of monensin e.g., in animal feed. A representative example of an LFA strip device is illustrated schematically in FIG. 1. The LFA strip device includes a sample pad onto which a liquid sample, e.g. extracted from animal feed (referred to herein as a “feed sample”) is applied, a conjugate pad that is loaded with relevant antibodies for the competition assay and a membrane surface having a test line and a control line loaded with relevant antigens for the competition assay. The liquid sample flows from the sample pad through the conjugate pad, allowing interaction of the liquid sample with the antibodies loaded onto the conjugate pad. The liquid sample then flows further onto the membrane surface, across the test line and the control line, allowing interaction of the liquid sample/conjugate mixture with the antigens loaded onto the test and control lines.

The LFA is based on competition between monensin in the liquid sample and monensin immobilized on the test line of the LFA strip device for binding to the anti-monensin antibody conjugate loaded onto the conjugate pad. The anti-monensin specific antibody (referred to herein as the “M antibody”) is conjugated to a detectable label such that binding of the M antibody conjugate to monensin immobilized on the test line results in a detectable signal. Thus, binding of the M antibody conjugate to monensin in the liquid sample reduces the amount of free M antibody conjugate available to bind to the immobilized monensin at the test line on the membrane surface, leading to a consequent reduction in the amount of detectable signal the develops at the test line.

A control antibody conjugate (referred to herein as the “C antibody”) is also loaded onto the conjugate pad and the control test line is loaded with the antigen to which the C antibody binds. Binding of the C antibody conjugate to its immobilized antigen results in a detectable signal at the control line on the membrane surface. This serves as an internal control for proper flow of the liquid sample along the LFA strip device. Additionally, the test and control lines can be read using an LFA strip device reader that detects and quantitates the detectable signal, thereby allowing for quantitative results for the assay.

Various aspects of the disclosure are described in further detail in the subsections below and in the Examples. The description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, databases and other references cited in this application are herein incorporated by reference in their entirety as if each individual publication, patent, patent application, database or other reference were specifically and individually indicated to be incorporated by reference.

### LFA Strip Device

Preparation of a representative example of an LFA strip device of the disclosure is described in detail in Example 2.

The material of the sample pad of the device is selected for its porosity, absorbency, width, wicking and other properties that allow for application of a liquid sample such that the sample is absorbed into the pad while allowing for efficient flow of the sample from the sample pad to the conjugate pad. In one embodiment, the sample pad is a polyester fiber pad. In one embodiment, the sample pad is a polyester Grade 6614 pad (Ahlstrom Munksjö, Helsinki, Finland) comprised of polyester fibers and a binder, with a basis weight of 75 g/m², a caliper of 0.42 mm, a wicking rate of 5 s/2 cm and a water absorption of 57 mg/cm², although pads of comparable properties are also suitable. The volume of feed sample used in the test is such that it will allow flow of the liquid from the sample pad to the conjugate pad based on the wicking and water absorption properties of the pad (typically X ul to X ul of feed sample). The sample pad is positioned in the device such that it makes physical contact with the conjugate pad such that liquid applied to the sample pad can flow into the conjugate pad via the junction of physical contact. The sample pad also is positioned in the device such that it does not make direct physical contact with the membrane surface; rather, the conjugate pad is positioned between the sample pad and the membrane surface.

The conjugate pad of the device is positioned in physical contact with the sample pad such that liquid applied to the sample pad can flow into the conjugate pad via the junction of physical contact. The conjugate pad also is positioned to makes physical contact with the membrane surface such that liquid from the conjugate pad flows onto the membrane surface via the junction of physical contact. The material of the conjugate pad also is selected for its porosity, absorbency, width, wicking and other properties to allow for inflow of the liquid sample from the sample pad, as well as outflow of the liquid sample onto the membrane surface. In one embodiment, the conjugate pad is a glass pad. In one embodiment, the conjugate pad is a chopped glass pad. In one embodiment, the conjugate pad is a microfiber glass pad. In one embodiment, the conjugate pad is a glass Grade 8951 pad (Ahlstrom Munksjö, Helsinki, Finland) comprised of chopped glass and a binder, with a basis weight of 75 g/m², a caliper of 0.38 mm, a wicking rate of 3 s/2 cm and a water absorption of 63 mg/cm², although pads of comparable properties are also suitable.

The membrane surface of the device is positioned in physical contact with the conjugate pad such that liquid from the conjugate pad flows onto the membrane surface, via the junction of physical contact, and across the test and control lines on the membrane surface. The membrane surface also is positioned in the device such that it does not make direct physical contact with the sample pad; rather, the conjugate pad is positioned between the sample pad and the membrane surface. The material of the membrane surface is selected for its ability to be loaded with the antigens that are immobilized at the test and control lines, as well as its porosity, absorbency, wicking and other properties pertaining to flow of the liquid sample across it. In one embodiment, the membrane surface comprises a nitrocellulose membrane. In one embodiment, the membrane surface is a 25 mm CN95 nitrocellulose membrane (Sartorius, Gottingen, Germany), although membranes of comparable properties are also suitable.

The sample pad, conjugate pad and membrane surface can be assembled with additional backing and/or wicking pads or membrane to provide structure and stability to the LFA strip device, as described in Example 2. Additionally, the LFA strip device can be assembled into a housing (e.g., plastic housing), such as a card or stick (dipstick) that allows for application of a liquid sample (e.g., using a pipette or dropper) onto the sample pad or dipping of the device into a liquid sample such that the liquid sample comes into contact with the sample pad.

The conjugate pad is loaded with a mixture of the anti-monensin (M) antibody and the control (C) antibody, each of which is detectably labeled. Typically, the M antibody conjugate comprises 90-95% of the applied antibody mixture and the C antibody conjugate comprises 5-10% of the applied mixture.

The concentration of M antibody loaded onto the conjugate pad can be adjusted to adjust the sensitivity of detection of monensin in the liquid sample. In various embodiments, the concentration of M antibody loaded onto the conjugate pad is 20-160 ug/mL, 20-120 ug/mL, 20-80 ug/mL, 20-60 ug/mL or 25-50 ug/mL. In one embodiment, the concentration of M antibody loaded onto the conjugate pad is 30-40 ug/mL. In one embodiment, the concentration of M antibody loaded onto the conjugate pad is 30 ug/mL. As described in Example 3, M antibody loaded onto the conjugate pad at a concentration of 30 ug/mL was sufficient to effectively detect monensin in a liquid feed sample down to a level of detection of as low as 2 ppm (2 gm/ton). In various embodiments, the level of detection is as low as 10 ppm, 9 ppm, 8 ppm, 7 ppm, 6 ppm, 5 ppm, 4 ppm, 3 ppm or 2 ppm.

The M antibody specifically binds to monensin (also known in the art as Monensin A, CAS 17090-79-8). As used herein, an antibody having “specific” binding to monensin includes antibodies that bind to monensin derivatives that share the same epitope to which the antibody binds on monensin. In one embodiment, the M antibody is a monoclonal antibody (mAb). In one embodiment, the M antibody is not a polyclonal anti-monensin antibody preparation. In one embodiment, the M antibody is a monoclonal antibody (mAb) that binds monensin and may also bind monensin derivatives that share the epitope to which the mAb binds. A non-limiting example of a suitable M antibody is a commercially available mouse anti-monensin mAb (Creative Diagnostics, Cat. No. HMABPY056, referred to herein as mAb HMABPY056), although other mAbs with similar binding properties (e.g., that cross-compete with HMABPY056 for binding to monensin) are also suitable for use. Thus, in one embodiment, the M antibody is mAb HMABPY056. In another embodiment, the M antibody is an anti-monensin-specific mAb that cross-competes with HMABPY056 for binding to monensin.

The C antibody is an antibody, preferably a monoclonal antibody (mAb), that binds a control antigen and that does not cross-react with monensin. A non-limiting example of a suitable C antibody is a commercially available goat anti-chicken IgY mAb (Lampire, Cat. No. 7455207), for use with a chicken IgY control antigen, although other mAbs that bind other control antigens are also suitable. The chicken IgY antigen to which the C antibody binds is also commercially available (Lampire, Cat. No. 9401400). In other embodiments, the C antibody is an anti-mouse IgG mAb, for use with a mouse IgG control antigen.

Antibodies used as the M and C antibodies typically are stored under sterile conditions (e.g., sterile saline) and have a purity that allows for consistency in their use in the LFA devices and methods of the disclosure. For example, in various embodiments, the M and/or C antibody is a monoclonal antibody having at least 90% purity and more preferably at least 95% or greater purity with respect to the presence of other proteins in the preparation.

The M antibody and C antibody are each labeled with a detectable label, typically with the same detectable label. The detectable label allows for visual detection of the test line and/or the control line when a conjugated antibody binds to its antigen at the test or control line. In one embodiment, the detectable label comprises gold nanoparticles. For example, the antibodies can be conjugated to gold nanoparticles using commercially available BioReady Gold Nanoshells (NanoComposix, Cat. No. GSXR150-100M), as described in Example 2. While a colloidal gold-based detectable label (e.g., gold nanoparticles) is preferred for ease of use and detection, other detectable labels suitable for use in lateral flow assays are known in the art and can be used in the LFA methods and devices of the disclosure, non-limiting examples of which include time-resolved fluorescent nanobeads, fluorescent submicrospheres and quantum dots (see e.g., Hu et al. (2017) Biosensors and Bioelectronics, 91:95-103).

The test line of the surface membrane is loaded with monensin as an antigen such that the monensin is immobilized at (i.e., affixed to) the test line. Typically, monensin is linked to a carrier protein to facilitate immobilization at the test line. In one embodiment, the carrier protein is an albumin, forming a monensin-albumin conjugate as the antigen used at the test line. Non-limiting examples of suitable albumins that can be used as the carrier protein include serum albumin (e.g., bovine serum albumin (BSA) or human serum albumin (HSA)), ovalbumin, alactalbumin or conalbumin. In one embodiment, the monensin antigen is BSA-monensin. A suitable BSA-monensin reagent, having known purity and consistency of use, is commercially available (Creative Diagnostics, Cat. No. DAGA-050B).

Similarly, the control line of the surface membrane is loaded with the antigen to which the C antibody binds such that the control antigen is immobilized at (i.e., affixed to) the control line. When anti-chicken IgY is used as the C antibody, a suitable chicken IgY antigen reagent is commercially available (Lampire, Cat. No. 9401400). In other embodiments, the C antibody is an anti-mouse IgG and the control antigen is a mouse IgG. Suitable reagents are commercially available in the art.

Loading of the monensin antigen and the control antigen at the test and control lines, respectively, to thereby immobilize them at those lines, is described in detail in Example 2. Preferably, an aerosol dispenser, such as a BioDot AirJet™ Nanoliter Aerosol Dispenser or equivalent, is used to “stripe” the test and controls line onto the membrane surface, for example using the parameters set forth in Example 2.

### Liquid Sample Preparation

To detect monensin using the LFA methods and devices of the disclosure, the monensin must be in a liquid sample that can be applied to the sample pad of the LFA strip device. Thus, for solid animal feed, a liquid extraction must be performed using a liquid extraction buffer. Preparation of liquid samples from animal feed and extraction buffers useful therefor are described further in Examples 1-3.

The liquid extraction buffer can be an aqueous buffer, an organic solvent buffer or a buffer combining aqueous and organic solvents. Since monensin is more soluble in organic solvents, the use of an extraction buffer that includes an organic solvent may be preferred for most efficient extraction. Alternatively, an aqueous extraction buffer (i.e., lacking any organic solvents) may be preferred for ease of use. In one embodiment, the extraction buffer comprises an alcohol, non-limiting examples of which include methanol, ethanol, butanol and isopropyl alcohol. In one embodiment, the extraction buffer comprises ethanol, typically at a concentration of at least 10%. The extraction buffer can include other components including buffering agents and surfactants. In one embodiment, the extraction buffer comprises phosphate buffered saline (PBS) with 0.5% Tween-20 and 10% ethanol. In one embodiment, the extraction buffer is phosphate buffered saline (PBS) with 1% Tween-20.

The liquid sample can be prepared from any type of animal feed suspected of containing monensin, including cattle, poultry, and equine feed. Typically, an aliquot (e.g., 5 grams) of animal feed is combined with an aliquot (e.g., 30 mL, or at approximately equivalent ratios) of liquid extraction buffer, the mixture may be allowed to soak (e.g., soaked for a specified time), and then is vortexed and allowed to settle. Extraction time is typically 5-20 minutes. As demonstrated in Example 3, an extraction time of 5 minutes using an extraction buffer of PBS with 0.5% Tween-20 and 10% ethanol was sufficient to allow for detection of monensin in the liquid feed sample down to a lower level of detection of approximately 6 ppm.

### Methods of Detecting Monensin

The LFA strip device and the liquid sample (e.g., liquid feed sample) are used in the methods of the disclosure for detecting monensin by applying the liquid sample to the sample pad of the LFA strip device thereby allowing the liquid sample to flow into the conjugate pad and along the membrane surface (thus crossing the test and control lines), developing the LFA strip device for at least 3 minutes (e.g., 5-10 minutes), and visually reading or quantitatively measuring the LFA strip device (i.e., the detectable signal at the test and control lines) to thereby detect the presence of monensin in the liquid sample. As demonstrated in Example 5, three minutes of development time is sufficient for accurate reading of the LFA strip device, although longer development times can be used accurately as well.

The liquid sample can be applied to the sample pad by dropping the liquid onto the pad (e.g., using a pipette or dropper) or by dipping the LFA strip device (e.g., dipstick) into the liquid sample such that the liquid comes into contact with the sample pad of the device. Typically, it requires 80-100 uL (e.g., 80 uL) of feed sample to adequately flow across the sample pad into the conjugate pad and sample for visual or quantitative reading.

Presence of the monensin in a test feed sample results in a decrease in the intensity of the detectable signal at the test line, relative to a liquid sample that lacks any monensin. The test line of the LFA strip device can be read qualitatively (e.g., by visual inspection) or quantitatively (e.g., by reading in a quantitative reader). For example, the intensity of the test line for a test animal feed sample can be compared to the intensity exhibited by a sample known to contain no monensin (0 ppm) and/or to a sample known to contain a known amount of monensin (e.g., 10 ppm or greater). As described herein, since the assay is a competitive LFA, the detectable signal at the test line decreases as the concentration of monensin increases in a sample. In one embodiment, a control LFA strip device(s) can be used with a sample(s) having a known amount of monensin, wherein the control LFA strip device is run in parallel with the test animal feed sample. Additionally or alternatively, the intensity of the test line for a test animal feed sample can be compared to a standardized test line intensity(ies) that indicates, for example, no monensin present and/or a known specified amount of monensin present. Such standardized test line intensities for specified amounts of monensin can be provided along with the LFA strip device for visual comparison purposes, e.g., a picture of the levels of intensities that represent specified amounts of monensin that the end user of the device compares to the intensity that develops for a test feed sample.

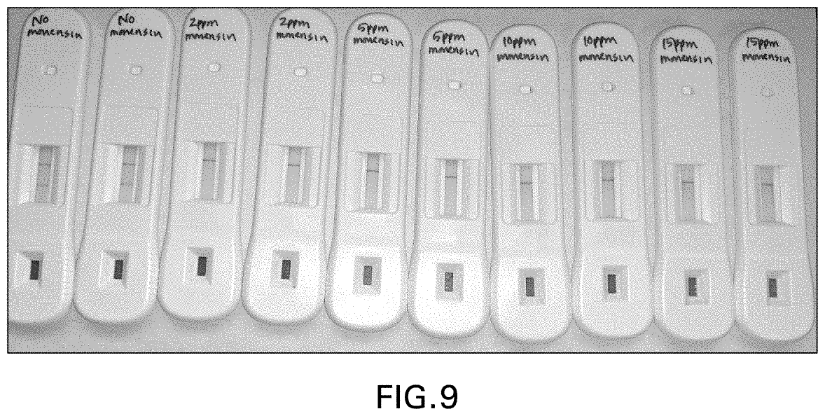

The LFA strip devices of the disclosure are highly sensitive for detection of monensin, having the ability to detect monensin down to a lower limit of approximately 2 ppm (see Example 3). The lower limit of detection (sensitivity) can be expressed in parts per million (ppm), wherein 1 ppm equals 1 gram monensin per 1 million grams (1000 kg) of animal feed. The lower limit of detection (sensitivity) also can be expressed in grams monensin/ton of animal feed (gm/ton). Since there are 2204 pounds in 1000 kg, and there are 2000 pounds per ton, 10 ppm corresponds to 9 gm/ton. Likewise, 12 gm/ton corresponds to 13.2 ppm, 6 gm/ton corresponds to 6.6 ppm and 3 gm/ton corresponds to 3.3 ppm. As described in Example 3 and further shown in FIG. 9, the LFA strip device and method of the disclosure can detect monensin in the liquid sample down to a concentration of as low as approximately 6 ppm (6 gm monensin/ton of animal feed) and even as low as 2 ppm. In various embodiments, the LFA strip device and methods have a lower limit of sensitivity of 6-10 ppm, 6.6-10 ppm or 6-9 gm/ton of feed for detection of monensin. In various embodiments, the LFA strip device and methods have a lower limit of sensitivity of 2 ppm, 3 ppm, 4 ppm, 5 ppm, 6 ppm, 6.6 ppm, 7 ppm, 8 ppm, 9 ppm or 10 ppm for detection of monensin. In various embodiments, the LFA strip device and methods have a lower limit of sensitivity of 2 gm/ton, 3 gm/ton, 4 gm/ton, 5 gm/ton, 6 gm/ton, 7 gm/ton, 8 gm/ton, 9 gm/ton, 10 gm/ton, 11 gm/ton or 12 gm/ton for detection of monensin.

For quantitative results, the LFA strip device can be read in a lateral flow assay reader that detects and quantifies the detectable label used (e.g., gold nanoparticles), for example by measuring the optical density (OD) at the test line. Such LFA readers are commercially available, non-limiting examples of which include the Leelu™ Reader (Lumos Diagnostics; Sarasota, Fla.), as well as LFA reader systems by GenPrime (Spokane, Wash.), Abingdon Health (York, UK) and NOW Diagnostics (Springdale, Ark.).

### EXAMPLES

The following examples are intended to provide illustrations of the application of the present invention. The following examples are not intended to completely define or otherwise limit the scope of the invention.

### Example 1: Detection of Monensin in Feed Samples by ELISA

In this example, preparatory studies were performed to determine whether monensin could be detected in animal feed using an anti-monensin antibody in an inhibition ELISA assay. Briefly, in the inhibition ELISA assay, the concentration of monensin was detected by signal interference. A capture antibody (sheep anti-monensin) was coated onto ELISA plates. During the incubation, a known amount of labeled monensin was combined with dilutions of test samples or standards, which contained unlabeled monensin (if present). These labeled/unlabeled monensin mixtures were added to the coated, blocked plates. The labeled monensin and the unlabeled test sample/standard monensin competed for binding to the capture antibody. This means the more unlabeled monensin there was in the mixture, the less labeled monensin could bind to the capture antibody. Following incubation, the plates were washed, and a substrate was added. The substrate changed color in the presence of the label (horseradish peroxidase [HRP] enzyme). Greater color change resulted from higher levels of the labeled monensin present, indicating lower levels of unlabeled monensin in the test sample. Lower amounts of color change (inhibition of signal) resulted from lower levels of labeled monensin, indicating higher levels of unlabeled monensin in the test sample.

### Determination of Antibody and Antigen Dilutions

An initial assay was run to determine appropriate levels of sheep anti-monensin antibody (Fitzgerald Industries, catalog number 20-1215, lot P17122113) and HRP-labeled monensin (Fitzgerald Industries, catalog number 80-1221, lot C19120303) required to result in a signal (color change) that will allow for detection of unlabeled monensin. The results of the dilution analysis are shown in Table 1:

TABLE 1
Determination of capture Antibody and Labeled Antigen Dilutions

| | Capture Antibody (sheep anti-monensin) | | | | | | | | | | | |
|---|---|---|---|---|---|---|---|---|---|---|---|---|
| *M | 1:500 | | 1:1000 | | 1:2000 | | 1:4000 | | 1:8000 | | 1:10,000 | |
| No M | 0.043 | 0.0427 | 0.0582 | 0.0422 | 0.042 | 0.0421 | 0.042 | 0.0427 | 0.0432 | 0.043 | 0.042 | 0.044 |
| 1:100 | 2.5856 | 2.4381 | 2.2509 | 2.0517 | 1.5242 | 1.5332 | 0.9482 | 0.9716 | 0.5253 | 0.5549 | 0.4593 | 0.5074 |
| 1:200 | 1.99 | 1.8494 | 1.587 | 1.5521 | 1.1755 | 1.1687 | 0.7242 | 0.7407 | 0.425 | 0.4156 | 0.3723 | 0.3644 |
| 1:400 | 1.1645 | 1.1545 | 0.9313 | 0.9839 | 0.7216 | 0.72 | 0.4564 | 0.4488 | 0.2655 | 0.2686 | 0.2416 | 0.2523 |
| 1:800 | 0.732 | 0.7507 | 0.5983 | 0.5876 | 0.4421 | 0.4385 | 0.2847 | 0.2959 | 0.1695 | 0.1764 | 0.1601 | 0.1635 |
| 1:1600 | 0.4459 | 0.412 | 0.3555 | 0.3329 | 0.2652 | 0.2633 | 0.1785 | 0.1752 | 0.1216 | 0.1194 | 0.1105 | 0.1066 |
| 1:3200 | 0.237 | 0.2314 | 0.203 | 0.2031 | 0.1553 | 0.1606 | 0.1278 | 0.1165 | 0.0842 | 0.0829 | 0.0775 | 0.0778 |
| 1:6400 | 0.1463 | 0.1429 | 0.1279 | 0.1264 | 0.1055 | 0.1039 | 0.0814 | 0.0877 | 0.0652 | 0.0652 | 0.0607 | 0.0617 |

*M = labeled monensin dilution

The results indicated that a capture antibody dilution of 1:1000 and a labeled monensin dilution of 1:200 were appropriate, with an O.D. reading of approximately 1.5 (shown in bold).

### Detection of Unlabeled Monensin

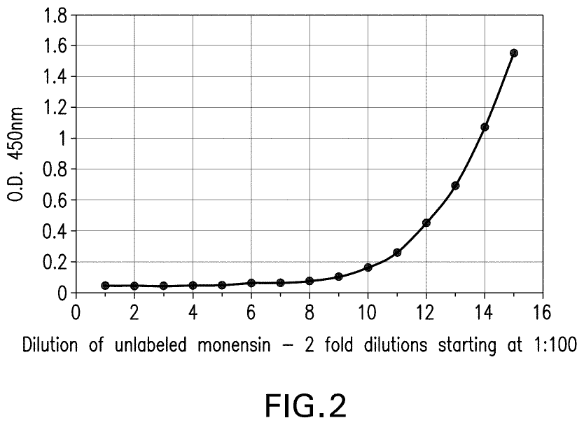

The determined conditions were used to evaluate the suitability of the assay to detect unlabeled monensin (Invitrogen, catalog number 00-455-51). For this assessment, plates were coated with sheep anti-monensin at 1:1000 dilution. Mixtures of labeled monensin (1:200 final dilution) and unlabeled monensin (various concentrations) were combined. After washing and blocking the coated plate, the mixtures of labeled/unlabeled monensin were added to plate wells. After incubation and washing, the HRP substrate was added. Plates were read for color development after stopping the reaction. The results are shown in FIG. 2. Since this is an inhibition ELISA, the lack of signal indicates high levels of monensin present; higher signal indicates lower levels of monensin present. The results demonstrated that under these conditions, monensin can be quantitated in a range of approximately 0.5-50 ng/mL.

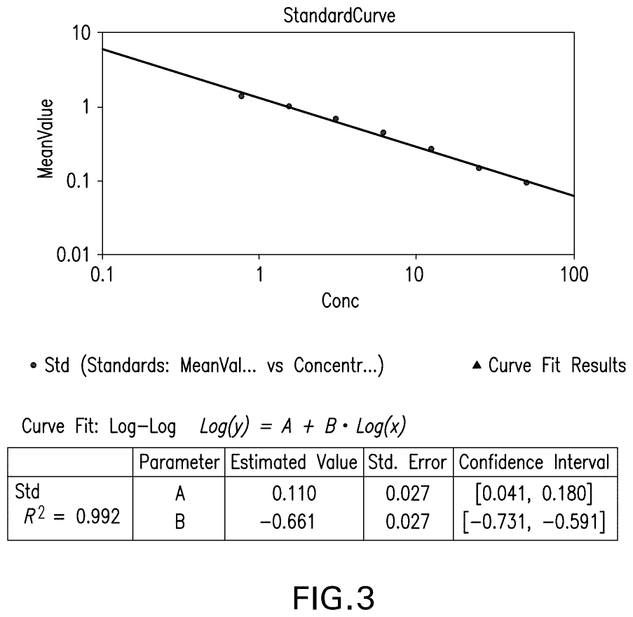

A second assay was completed with the monensin standard curve to determine the detectable monensin range more precisely. The results are shown in FIG. 3. The results confirmed a range of 0.781-50 ng/mL of monensin.

### Feed Extract Preparation

Feed extracts were prepared by combining 1 g aliquots of feed (with or without monensin) with 5 mL of ethanol or phosphate buffered saline (PBS). Samples were vortexed vigorously and incubated at room temperature until the feed settled. The liquid was removed from each sample and centrifuged at ˜13,000×g for 10 minutes to pellet debris. The clarified extracts were used as test samples in the inhibition ELISA.

### Detection of Monensin in Feed Extracts

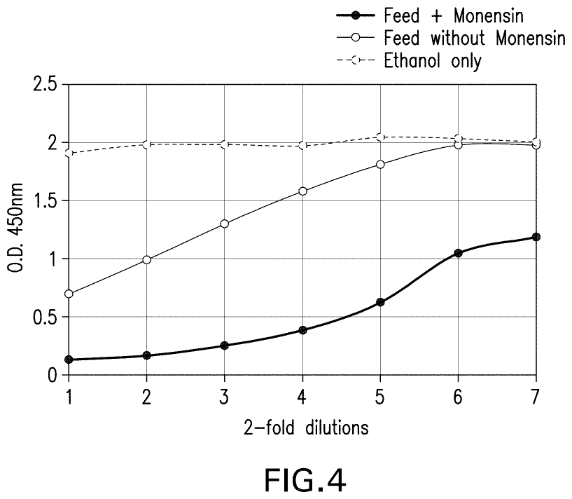

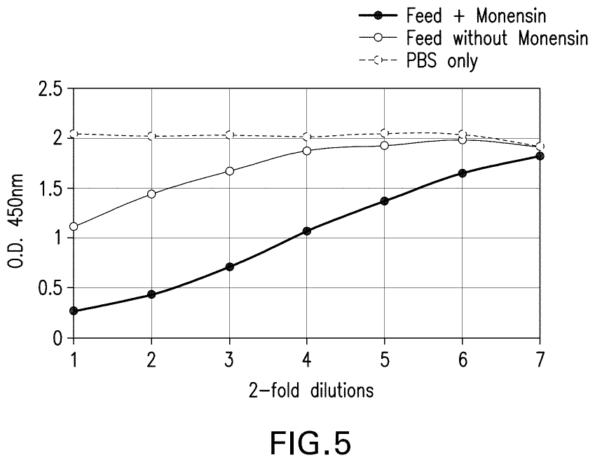

Feed extracts prepared as described above were assessed in the inhibition ELISA as described above. Dilutions of clarified extracts were combined with a known, standard amount of labeled monensin. As controls, ethanol alone or PBS alone were tested at the same dilutions as the extracts. Using the monensin standard curve, the concentration of monensin in each extract was determined. The results are shown in FIG. 4 and FIG. 5 (for ethanol and aqueous extracts, respectively) and summarized below in Table 2:

TABLE 2
Detection of Monensin in Feed Extracts

| | Amount of Monensin |
|---|---|
| Sample | Detected (ng/mL) |
| Feed WITH monensin, ethanol extraction | 3764.9 |
| Feed WITHOUT monensin, ethanol extraction | 215.4 |
| Ethanol only | Not detected |
| Feed WITH monensin, PBS extraction | 164.8 |
| Feed WITHOUT monensin, PBS extraction | 19.0 |
| PBS only | Not detected |

The result showed that monensin was detected in all extracted samples, but appeared to be extracted from feed more efficiently using ethanol as compared to PBS. This is as expected, because monensin is more soluble in organic solvents than in aqueous solution. The results of the competitive ELISA assay demonstrate the specificity of the antibody to monensin, and define the limits of monensin detection that can be achieved in a competitive type of assay and that potentially could be translated to the lateral flow methodology.

### Example 2: Detection of Monensin in Feed Samples by Lateral Flow Assay

In this example, a quantitative competitive lateral flow assay (LFA) was developed for detection of monensin in feed samples. The LFA device is illustrated schematically in FIG. 1. Briefly, a conjugate antibody preparation comprising anti-monensin antibody conjugated to gold nanoparticles is loaded onto the conjugate pad of an LFA device. A small amount of a control antibody (anti-chicken IgY) is also included in the mixture loaded onto the conjugate pad. When the extracted feed sample is applied to the sample pad of the device, the sample mixes with the anti-monensin conjugate and the sample/conjugate mixture migrates and crosses the test line of the LFA device. The test line has BSA-monensin immobilized on it and thus any anti-monensin conjugate with free binding sites (i.e., that have not bound to monensin in the feed sample) is captured by the test line, forming a visibly observable line. As more monensin in the sample competes for binding to the anti-monensin conjugate, less of the conjugate is free to bind the BSA-monensin on the test line. Thus, if no monensin (or levels below detection) is present in the feed sample, the test line is visually observed at its highest intensity. However, if monensin is present in the feed sample (at a level above the detection limit), it inhibits the binding of the conjugate to BSA-monensin on the test line and thus the intensity of the test line diminishes, proportional to the amount of monensin in the sample. A control line is also present, loaded with the antigen (chicken IgY) to which the control antibody (anti-chicken IgY) binds, thus serving as a positive control for the device.

### Conjugate Pad Preparation

A commercially available chopped glass pad (Ahlstrom-Munksjo; Grade 8951), cut into 10 mm×300 mm strips, was used for the conjugate pad. 2 mL of conjugate pad treatment buffer (PBS, 0.5% BSA, 0.5% Tween-20, pH 7.4) at room temperature was applied to each conjugate pad and allowed to soak at room temperature for 30 minutes. Treated conjugate pads were dried on drying trays in a 37° C. oven for 2 hours. After drying, the conjugate pads were stored with desiccant until use.

### Striping of Test and Control Lines

A commercially available CN95 nitrocellulose membrane, 25 mm, (Sartorius; 1UN95ER100025NT) and a commercially available backing card, 73.5 mm (Lohmaann; GL-57312) were used for the portion of the LFA device loaded with the test and control lines. The center lining and membrane were removed from the backing card and the CN95 membrane was laminated onto the backing card. The liner was used to smooth the CN95 membrane onto the backing card to ensure proper adhesion.

For the test line and control line, monensin-BSA conjugate (Creative Diagnostics; DAGA-050B) and goat anti-chicken IgY (Lampire; 7455207) were used, respectively. Monensin-BSA was diluted to 2 mg/ml and anti-chicken IgY was diluted to 0.5 mg/ml in 1×PBS, 0.5% sucrose, pH 7.4. The test line (TL) was positioned in the center of the membrane, with the control line (CL) positioned 5 mm downstream from the TL with respect to the direction of flow of the liquid sample applied to the sample pad (i.e., the CL is positioned, relative to the TL, such that the liquid sample first flows over the TL and then over the CL).

A BioDot AirJet™ Nanoliter Aerosol Dispenser was used to “stripe” the test and controls lines onto the membranes, according to the pattern settings shown below in Table 3:

TABLE 3
BioDot Pattern Settings for Striping of Test Line and Control Line

| Parameter | Setting |
|---|---|
| Active | Yes |
| Shape | Line |
| Z enabled | Yes |
| Length (mm) | 310 |
| Polarization | Yes |
| Speed (mm/s) | 40 |
| Acceleration (mm/s2) | 1000 |
| X Start | 0 |
| Y Start | TL and CL should be |
| | 3 mm off center of |
| | Membrane |
| Z Up (mm) | 0 |
| Z Down (mm) (adjust as needed) | 54.5 |
| Syringe Pump 3 (Dispense Rate ul/cm) | 0.7 |
| Syringe Pump 4 (Dispense Rate ul/cm) | 0.7 |

After striping of the TL and CL on the membranes, they were dried in a 37° C. oven for 1 hour. After drying, the membranes were stored with desiccant until use.

### Gold Nanoshell Conjugation Procedure

The anti-monensin detector antibody (Creative Diagnostics; HMABPY056) was conjugated to gold nanoparticles using BioReady Gold Nanoshells (GNS), carboxyl, 120 nm particles (NanoComposix; GSXR150-100M).

To make a stock preparation of GNS, beads were mixed on a rotator for 5-10 minutes, bath sonicated for 5 seconds and vortexed twice for 5 seconds. Beads were aliquoted to a final volume of 1 mL×1 tube.

For activation, solutions of EDC (1-ethyl-3-(3-dimethylaminopropyl)carbodiimide hydrochloride; ThermoScientific; Cat. No. 22980) and Sulfo-NHS (N-hydroxysulfosuccinimide; ThermoScientific; Cat. No. 24510) were made fresh and used within 10 minutes of preparation. EDC and Sulfo-NHS solutions were made to 10 mg/mL with deionized water.

8 uL EDC was added to an aliquot of GNS particles and vortexed briefly. 16 uL Sulfo-NHS was immediately added to the (EDC added) aliquot of GNS, vortexed briefly and returned to a rotator. When all tubes were activated, each tube was sonicated and vortexed, the tubes were rotated for 30 minutes at room temperature, bath sonicated for 5 seconds and vortexed for 5 seconds two times. The beads were then centrifuged at 2000 g for 7 minutes at 20° C., the supernatants aspirated and 1 mL of 1× Reaction Buffer (0.01 PBS, 0.5% PEG, pH 7.4) was added to each pellet of the activated GNS particles, followed by bath sonication for 5 seconds and vortexing twice for 5 seconds.

For antibody coupling, stock antibody at 40 ug/mL was added to each tube of activated beads, bath sonicated for 5 seconds and vortexed twice for 5 seconds. The mixture was rotated for 30 minutes at room temperature, bath sonicated for 5 seconds and vortexed twice for 5 seconds. To quench the coupling reaction, 5 uL of 50% ethanolamine (in deionized water) was added to each tube, bath sonicated for 5 seconds and vortexed twice for 5 seconds. The tubes were rotated for 10 minutes at room temperature and the beads centrifuged at 2000 g for 7 minutes at 20° C. The supernatants were aspirated and the beads resuspended in 1 mL 1× Reaction Buffer, bath sonicated for 5 seconds and vortexed twice for 5 seconds. The beads were again centrifuged at 2000 g for 7 minutes at 20° C., supernatants aspirated, the beads resuspended in 1 mL 1× Reaction Buffer, bath sonicated for 5 seconds and vortexed twice for 5 seconds.

For final resuspension and measurement of absorbance, the beads were centrifuged again at 2000 g for 7 minutes at 20° C., supernatants aspirated, the beads resuspended in 0.5 mL Conjugate Dilution (0.5×PBS, 1% BSA, 1% Tween-20, 0.05% ProClin 300, pH 8.0), bath sonicated for 5 seconds and vortexed twice for 5 seconds. Absorbance (OD) was measured at Peak Wavelength according the GNS manufacturer's instructions, 5 ul conjugate in 495 ul Conjugate Diluent (typically 810-830 nm). The conjugated anti-monensin antibody was stored at 4° C. until use.

### Conjugate Spraying

Conjugate pads, prepared as described above, were sprayed with the anti-monensin conjugate preparation. The preparation was also spiked with a small amount (5-10%) of the control goat anti-chicken IgY antibody (Lampire; 9401400). The conjugate preparation was first bath sonicated for 5 seconds and vortexed for 5 seconds three times. Sucrose and Trehalose were added to the preparation at 10% and 5%, respectively, and the mixture vortexed until the sugars dissolved. The preparation was again bath sonicated for 5 seconds and vortexed for 5 seconds three times.

The anti-monensin conjugate preparation (including control antibody) was sprayed onto the prepared conjugate pad using a BioDot AirJet™ Nanoliter Aerosol Dispenser, according to the pattern settings shown below in Table 4:

TABLE 4
Biodot Pattern for Spraying Conjugate
Preparation onto Conjugate Pad

| Parameter | Setting | |
|---|---|---|
| Biodot Program | AirjetQuanti | |
| Syringe volume | 250 | μL |
| Length | 310 | mm |
| Polarization | Yes | |
| Speed | 40 | mm/s |
| Acceleration | 1000 | mm/s² |
| X Start | 5 | mm |
| Y Start (adjust as needed) | 7 | mm |
| Z Down (adjust as needed) | 59 | mm |
| Aperture | 1 | |
| Rate | 6 | μL/cm |

| | |
|---|---|
| PSI | 5 (at rest) |

After spraying of the conjugate pads, they were dried in a 37° C. oven for 1 hour. After drying, the conjugate pads were stored with desiccant until use.

### LFA Strip Preparation and Assembly

Absorbent/Wick pads (Whatman; Grade 470; 18 mm) were cut into 18 mm strips. The top lining was removed from a backing card (Lohmann; GL-57312; 73.5 mm) and the Whatman 470 wick pad was laminated flush with the top edge of the backing card. A liner was used to smooth the wick pad onto the backing pad to ensure proper adhesion.

To affix the conjugate pad and sample pad to the strip, the bottom 2 liners were removed from the backing card. The 10 mm Ahlstrom 8951 conjugate pad was laminated overlapping the bottom of the membrane 2 mm. A liner was used to smooth the conjugate pad onto the backing card to ensure proper adhesion. The 16 mm Ahlstrom 6614 sample pad was laminated flush with the bottom of the card. The assembled cards were cut into 4 mm strips using a Kinematic cutter. The assembled LFA strips were stored with desiccant until use.

### Sample Extraction and LFA Protocol

0.5 grams of feed sample was weighed out into a 5 mL tube and 2 mL of Extraction Buffer (PBS with 0.5% Tween-20 and 10% ethanol) was added. The mixture was vortexed for 5 seconds and let sit for 5 minutes. The sample extract was diluted, typically in a range of 1/20-1/50 prior to applying the sample extract to the LFA strip.

The LFA strip was laid on a flat surface and 100 ul of feed sample was applied to the sample pad of the strip. After 10-15 minutes, the strip was read in a Leelu Reader (Lumos Diagnostics). Alternatively, the feed sample can be placed in a tube or well and the LFA strip can be used as a dipstick to contact the sample pad with the feed sample.

### Example 3: Dose Responsiveness of Monensin Lateral Flow Assay

In this example, the LFA strips for detecting monensin were used with feed samples containing varying known amounts of monensin to examine the dose responsiveness of the assay and the time dependency of the feed extraction.

The LFA strips were prepared as described in Example 2, with the following reagent concentrations. For the test line (TL), monensin-BSA (Creative Diagnostics) in 1×PBS, 1% sucrose, was applied (0.7 ul/cm) at a concentration of 1.25 mg/mL per Example 2. For the control line (CL), 0.5 mg/mL chicken IgY was used. For the anti-monensin antibody (Creative Diagnostics), the antibody-GNS conjugate in GNS Diluent (0.5×PBS, 1% BSA, 1% Tw-20, 0.05% ProClin 300, pH 8.0) was sprayed on the conjugate pad (10 ul/cm) at a concentration of either 30 ug/mL or 40 ug/mL, referred to herein as Load 30 and Load 40, respectively per Example 2. The conjugate preparation applied to the conjugate pad also included 10% anti-chicken IgY as the positive control.

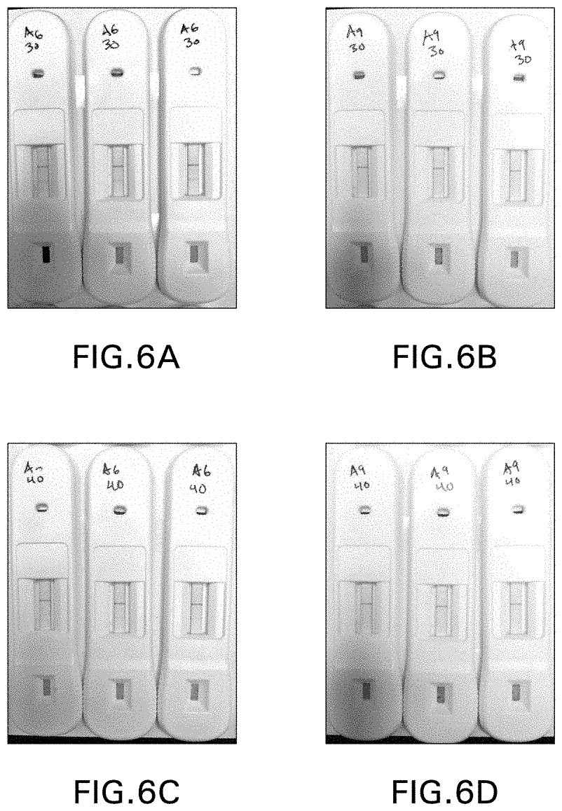

In a first set of experiments, feed samples (prepared as described in Example 2) known to contain either 6 g/ton (6 ppm) or 9 g/ton (9 ppm) of monensin were tested with LFA strips prepared with either 30 ug/mL (Load 30) or 40 ug/mL (Load 40) of anti-monensin conjugate. Each combination, i.e., Load 30 with 6 or 9 grams/ton and Load 40 with 6 or 9 grams per ton, was tested in triplicate for a total of 12 LFA strips compared. The results are shown in FIG. 6A-D. The results showed that for the 6 g/ton sample, the signal was stronger using the Load 30 concentration than the Load 40 concentration, whereas for the 9 g/ton sample, the signal was approximately equal with both concentrations. Accordingly, the Load 30 concentration was used for subsequent assays.

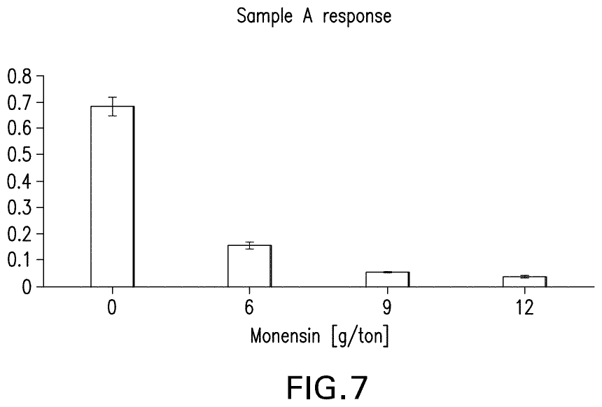

In a second set of experiments, feed samples spiked with 0, 6, 9 or 12 grams/ton of monensin were tested with Load 30 LFA strips and the intensity of the signal from the test line was quantitated using a Leelu Reader, as described in Example 2. The results are shown in FIG. 7, which demonstrates that increasing amounts of monensin in the sample led to decreasing intensity of the test line signal, as expected for a competitive lateral flow assay. Moreover, the LFA strips detected the monensin in a dose-responsive manner, showing the strips are sufficiently sensitive to distinguish between 6, 9 or 12 grams/ton of monensin in the feed sample.

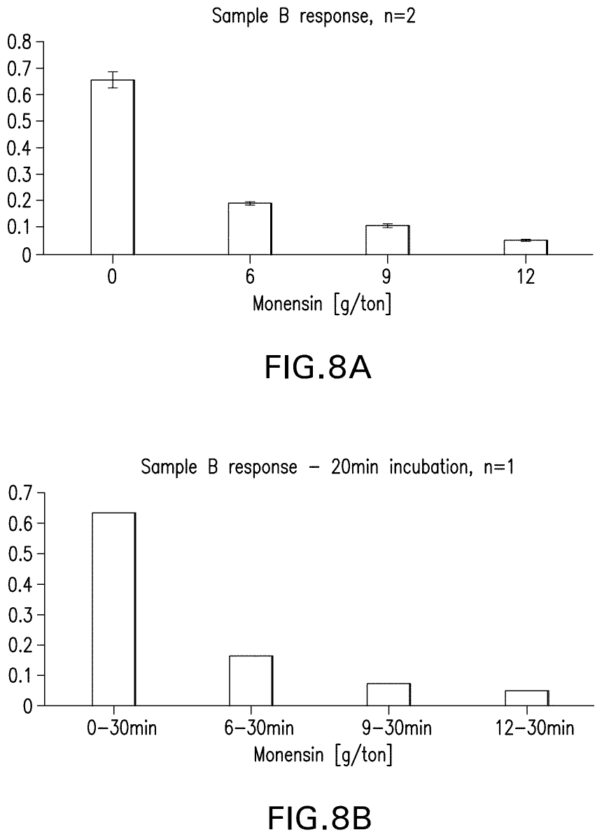

In a third set of experiments, feed samples spiked with 0, 6, 9 or 12 grams/ton of monensin were again tested with the Load 30 LFA strips, but samples extracted for 5 minutes were compared to samples extracted for 20 minutes. The results are shown in FIG. 8A (5 minutes) and FIG. 8B (20 minutes). The results demonstrate that the test line signal was consistent whether the samples were extracted for 5 minutes or 20 minutes. Thus, the results demonstrate that a 5 minute extraction was sufficient for accurate detection of monensin in the feed sample using the lateral flow assay.

In a fourth set of experiments, feed samples spiked with 0, 2, 5, and 10 ppm of monensin were tested in duplicate. The results are shown in FIG. 9, which demonstrates that increasing amounts of monensin in the sample led to decreasing intensity of the test line signal, as expected for a competitive lateral flow assay. Thus, the results demonstrate that the strips are sufficiently sensitive to distinguish between 0, 2, 5, and 10 ppm of monensin in the feed sample.

In summary, these experiments demonstrated successful use of the LFA strips to detect monensin in feed samples both qualitatively and quantitatively. Moreover, the results demonstrate that a concentration of 30 ug/mL of anti-monensin conjugate and a feed sample extraction time of 5 minutes are sufficient to allow for detection of monensin at a high degree of sensitivity (down to approximately 6 ppm). Furthermore, the results demonstrate that the LFA strips are capable of quantitatively distinguishing samples containing 0, 2, 5, 6, 9 or 12 g/ton (approximately 0-12 ppm) of monensin.

### Example 4: Analytical and Cross Reactivity Study

In this example, an analytical and cross reactivity study was performed to determine whether various ionophores, antibiotics, and feed additives other than monensin were detected by the monensin lateral flow assay. Samples were prepared by combining monensin-negative feed samples with high concentrations of the test substances. These samples were then extracted and assayed according to the test method as described in Examples 2 and 3. The results are shown below in Table 5:

TABLE 5
Analytical & Cross Reactivity Results

| | | % Replicates |
|---|---|---|
| | | with Positive |
| Test Substance | Sample Concentration | Result |
| Narasin (NAR) | 2 1bs. Monteban 45 per ton of feed | 0% |
| Salinomycin (SAL) | 1 lb. Biocox 60 per ton of feed | 0% |
| Tylosin (TYL) | 10 lbs. Tylovet per ton of feed | 0% |
| Ractopamine (RAC) | 17.62 lbs. Optigrid 45 per ton of feed | 0% |
| Amprolium (AMP) | 100 lbs. Corid per ton of feed | 0% |

The results demonstrated that the monensin lateral flow assay did not detect Narasin (NAR), Salinomycin (SAL), Tylosin (TYL), Ractopamine (RAC), or Amprolium (AMP) in feed samples at concentrations up to those listed in Table 5 above, thus confirming the lack of cross reactivity of the assay.

### Example 5: Time to Result Study

In this example, a time to result study was performed to determine the impact on test results of reading the test device for more or less than 5 minutes. Monensin-containing samples were extracted and assayed according to the test method as described in Examples 2 and 3, with the test device read at either 3 minutes, 5 minutes or 7 minutes. The results are shown below in Table 6:

TABLE 6
Time to Result Results

| | Read Time and % Replicates with Positive Result | | |
|---|---|---|---|
| Sample Tested | 3 minutes | 5 minutes | 7 minutes |
| High Negative | 0% | 0% | 0% |
| Low Positive | 100% | 100% | 100% |

The results demonstrated that the monensin lateral flow assay was accurate at all three read times tested, indicating that while the 5 minute read time is typical, the test is accurate both at shorter and longer read times as well.

### Example 6: Reproducibility and Repeatability Study

In this example, a study was conducted to determine the reproducibility of the monensin lateral flow assay by evaluating visually-interpreted results across multiple replicates of a high-negative and low-positive sample. Testing was performed by two different operators across three days, split between morning and afternoon runs.

Reproducibility is defined as the variation in interpreted results observed by a single person under the same conditions over the period of testing. Reproducibility results are shown below in Table 7:

TABLE 7
Reproducibility Results

| | % Replicates with Positive Result |
|---|---|
| Sample Tested | All Operators, Days and Time Points |
| High Negative | 0% |
| Low Positive | 100% |

The results in Table 7 demonstrated that the monensin lateral flow assay was reproducible.

Repeatability is defined as the variation of an entire study across multiple operators, days and times of testing. Repeatability results are shown below in Table 8, reporting results for Operators 1 & 2 across multiple days and time points, and results for Days 1, 2 & 3 across multiple operators:

TABLE 8
Repeatability Results

| | Read Time and % Replicates with Positive Result | | | | |
|---|---|---|---|---|---|
| Sample Tested | Operator 1 | Operator 2 | Day 1 | Day 2 | Day 3 |
| High Negative | 0% | 0% | 0% | 0% | 0% |
| Low Positive | 100% | 100% | 100% | 100% | 100% |

The results in Table 8 demonstrated that the monensin lateral flow assay was repeatable.

### Example 7: Sample Weight Study

In this example, a study was performed to determine how variations in the sample-weighing step might affect the monensin lateral flow assay results. A high-negative and low-positive sample were each extracted with three different sample weights, 4.0 grams, 5.0 grams or 6.0 grams of feed. The assay steps other than the sample-weighing step were performed according to the method as described in Examples 2 and 3. The results are shown below in Table 9:

TABLE 9
Sample Weight Results

| | Sample Weight and % Replicates with Positive Result | | |
|---|---|---|---|
| Sample Tested | 4.0 grams | 5.0 grams | 6.0 grams |
| High Negative | 0% | 0% | 0% |
| Low Positive | 100% | 100% | 100% |

The results demonstrated that the monensin lateral flow assay was accurate at all three sample weights tested, indicating that while 5 grams of sample feed is typical as a sample weight, the test is accurate both at lower and higher sample weights as well.

### Example 8: Sample Mixing Time Study

In this example, a study was performed to determine how variation in the duration of the sample mixing step might affect the monensin lateral flow assay results. A high-negative and low-positive sample were each prepared using five different sample mixing times, 0 seconds, 5 seconds, 10 seconds, 20 seconds and 60 seconds. The assay steps other than the sample mixing time were performed according to the method as described in Examples 2 and 3. The results are shown below in Table 10:

TABLE 10
Sample Mixing Time Results

| | Sample Mixing Time and % Replicates | | | | |
|---|---|---|---|---|---|
| | with Positive Result | | | | |
| | 0 | 5 | 10 | 20 | 60 |
| Sample Tested | Seconds | Seconds | Seconds | Seconds | Seconds |
| High Negative | 0% | 0% | 0% | 0% | 0% |
| Low Positive | 100% | 100% | 100% | 100% | 100% |

The results in Table 10 demonstrated that the monensin lateral flow assay was accurate at all five sample mixing times tested, indicating that while the 10-second sample mixing time is typical, the test is also accurate using shorter and longer sample mixing times.

1. A competitive lateral flow assay (LFA) strip device for detection of monensin in a liquid sample, the device comprising:
a sample pad;
a conjugate pad loaded with an anti-monensin-specific antibody (M antibody) conjugated to a detectable label and a control antibody (C antibody) conjugated with a detectable label; and
a membrane surface comprising a test line with immobilized monensin to which the M antibody binds and a control line with immobilized antigen to which the C antibody binds,
wherein a liquid sample applied to the sample pad flows first through the conjugate pad and then across the test line and control line of the membrane surface;
wherein binding of the M antibody to the test line results in a detectable signal and binding of the C antibody to the control line results in a detectable signal;
wherein presence of monensin in the liquid sample decreases the detectable signal at the test line relative to a sample lacking monensin; and
wherein the LFA strip device has a sensitivity of 10 ppm or less for detecting monensin in the liquid sample.

2. The LFA strip device of claim 1, which has a sensitivity of 2 ppm for detecting monensin in the liquid sample.

3. The LFA strip device of claim 1, which has a sensitivity of 6-10 ppm for detecting monensin in the liquid sample.

4. The LFA strip device of claim 1, wherein the sample pad is a polyester fiber pad.

5. The LFA strip device of claim 1, wherein the conjugate pad is a chopped glass pad.

6. The LFA strip device of claim 1, wherein the membrane surface comprises a nitrocellulose membrane.

7. The LFA strip device of claim 1, wherein the M antibody is conjugated to gold nanoparticles.

8. The LFA strip device of claim 1, wherein the immobilized monensin at the test line is BSA-monensin.

9. The LFA strip device of claim 1, wherein the C antibody is anti-chicken IgY or anti-mouse IgG antibody conjugated to gold nanoparticles and the immobilized antigen at the control line is chicken IgY or mouse IgG.

10. A method of detecting presence of monensin in animal feed, the method comprising:
(a) contacting the animal feed with a liquid extraction buffer to obtain a liquid sample;
(b) applying the liquid sample to the sample pad of the LFA strip device of any one of claims 1-12;
(c) developing the LFA strip device for at least 3 minutes; and
(d) visually reading or quantitatively measuring the LFA strip device to thereby detect presence of monensin in the animal feed.

11. The method of claim 10, wherein the liquid extraction buffer comprises an organic solvent.

12. The method of claim 11, wherein the organic solvent is an alcohol.

13. The method of claim 12, wherein the alcohol is ethanol.

14. The method of claim 10, wherein the liquid extraction buffer comprises phosphate buffered saline (PBS) with 0.5% Tween-20 and 10% ethanol.

15. The method of claim 10, wherein the liquid extraction buffer is an aqueous buffer.

16. The method of claim 10, wherein the liquid extraction buffer is phosphate buffered saline (PBS) with 1% Tween-20.

17. The method of any one of claims 10-16 wherein the animal feed is contacted with the extraction buffer for 20 minutes or less.

18. The method of claim 17, wherein the animal feed is contacted with the extraction buffer for 5 minutes.

19. The method of any one of claims 10-18, wherein the LFA strip device is read using a quantitative reader.
